(19) United States
(12) Patent Application Publication
(10) Pub. No.: US 2013/0230426 A1
(43) Pub. Date: Sep. 5, 2013

(54) DEVICE FOR COLD PLASMA STERILIZATION OF AN OBJECT, SUCH AS MEDICAL DEVICE, PARTICULARLY AN IMPLANT, AND METHOD OF USING THIS DEVICE

(71) Applicants: Jean-Marc Popot, Villers-Semeuse (FR); Marie-Paule Gelle, Reims (FR)

(72) Inventors: Jean-Marc Popot, Villers-Semeuse (FR); Marie-Paule Gelle, Reims (FR)

(73) Assignee: Universite de Reims Champagne Ardenne, Reims (FR)

(21) Appl. No.: 13/825,873

(22) PCT Filed: Sep. 23, 2011
(86) PCT No.: PCT/FR11/52199

§ 371 Date: May 13, 2013

(30) Foreign Application Priority Data
Sep. 24, 2010 (FR) 1057716

(51) Int. Cl. A61L 2/14

(52) U.S. Cl. CPC A61L 2/14
USPC 422/29; 422/186.29; 422/118; 422/119

(57) ABSTRACT

The cold plasma sterilization device for the sterilization of at least one object is placed in a container enclosing at least a gas or a gas mixture suitable for generating a cold plasma. The device includes a closed vessel in which a support for receiving the container is arranged. There is a device for subjecting the vessel to a secondary vacuum of the order of 10⁻³ to 10⁻⁷ millibar. There is an electron cyclotron resonance generator for generating, in the vessel, a flow of microwaves coupled to a magnetic field at least in a phase of initialization of the cold plasma. There is also a device for polarization of the support of the container.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The invention relates to a device for cold plasma sterilization of at least one object, such as a medical device, an implant or the like, placed in a container containing at least one gas or an appropriate gas mixture for generating a cold plasma. The invention also relates to a sterilization method implementing such a sterilization device.

The present invention is related to the field of the sterilization of objects, namely medical devices, such as surgical instruments, implants, even protection garments used in this medical field.

2. Description of Related Art Including Information Disclosed Under 37 CFR 1.97 and 37 CFR 1.98

In a known way, the cold plasmas are induced by subjecting a gas, or a gas mixture, to an electric field, traditionally generated at middle or high frequency, by operating at atmospheric pressure or at reduced pressures in the range of 10⁻¹ millibar, i.e. 10 Pascal (Pa). They have the peculiarity, depending on the gas or gas mixture being used, of generating a UV radiation and active species, such as atomic oxygen, between which a synergy operates so as to permit the destruction of bacterial strains.

This technology constitutes an interesting alternative for the traditional sterilization methods, as for example by moist heat as the autoclave, the use of ethylene oxide or ionizing rays such as gamma rays, when the latter do not prove satisfactory, whether from the point of view of their efficiency or of their eventual adverse effects on the objects or medical devices being treated.

Indeed, the sterilization by means of an autoclave or moist heat, though it defines a quick, efficient and cheap method, remains inappropriate for the treatment of objects made of polymer, while the ethylene oxide spreads inside the polymers and requires to let the treated surface desorb during at least 24 hours.

As regards the ionizing gamma radiations, though they are very efficient on the polymeric materials, they have nevertheless the major drawback of a risk of degradation of the chains, causing a modification of the characteristics of the material, because of the high radiation energy they diffuse.

Thus, the cold plasmas, the implementation of which can occur at a temperature lower than 80° C., even at room temperature, are particularly suited for sterilizing heat-sensitive objects or materials, which would risk to deteriorate under the action of the high temperatures that some traditional methods involve; the cold plasmas permit furthermore to avoid a number of other physical-chemical aggressions that can sometimes be observed with the latter, namely with objects made of polymeric materials.

Finally, the cold-plasma sterilization has the advantage of generating only gaseous effluents, which are both inoffensive for the operator and completely environment friendly.

Several prior-art documents describe methods or devices for sterilization that are based on the cold-plasma technology.

WO 00/72889 discloses a system and a method for plasma sterilization at low temperature, in which a gas is used that does not need to have an intrinsic sterilizing action, the latter resulting from the flowing of said gas through an electric field generated by microwaves, said gas comprising oxygen in molecular form and an atomic or molecular species capable of emitting an ultraviolet radiation after having been excited.

The sterilization occurs at a temperature lower than 50° C., placing the objects or materials involved inside a sterilization vessel connected to a vacuum pump and to a plasma source, and submitting them as such to a flow of said plasma.

WO 00/54819 relates to a method and a device for plasma sterilization, wherein the objects to be treated are placed in a sterilization vessel substantially at atmospheric pressure in which one or several non-biocide gas mixtures, at least one of which contains moisture, are introduced.

Then, a plasma producing active species is created from one of the gas mixtures by generating by means of a high-voltage supply an electric discharge between two electrodes placed in said sterilization vessel, and said plasma and said moisture are directly conveyed to the surface of the objects to be treated.

The gas mixture recommended by this document contains at least 10% oxygen and 10% nitrogen and is preferably formed of ambient air.

In fact, although these documents describe methods for cold-plasma sterilization and/or devices for their implementation, capable of efficiently treating objects by subjecting them directly to the flow of plasma, none of them deals with and permits to solve the problem of the conditioning of these objects, in which step a new contamination by bacterial strains is often observed.

The above-mentioned methods can therefore not be considered fully satisfactory, since they are limited to the sterilization itself of the objects, without providing a solution permitting to ensure the preservation of the sterile nature during and after the conditioning.

A solution that has been provided for solving this problem, and described namely in U.S. Pat. No. 4,321,232, consists in carrying out a sterilization on an object that is already conditioned.

The packaging used is of a porous nature, permitting the penetration of the plasma generated through traditional means outside this packaging.

It could however be observed that this solution neither provides full satisfaction, because it requires a much higher time for treating the object, in order to reach a degree of sterilization comparable to that achieved with a flow of plasma that is applied directly.

Indeed, the thickness of the membrane of the porous packaging impedes the diffusion of the ultraviolet rays until they reach the objects, and furthermore causes phenomena of recombination of the active species, which are then no longer capable of ensuring their role optimally.

From FR 2 850 280 of the same applicant is also known a method for cold-plasma sterilization of medical devices, implants or the like, wherein a sealed bag filled with gas is placed in a sterilization vessel. This bag is then subjected to an electric field that will induce a plasma inside the bag by acting on the pressure of the gas enclosed in said bag.

Finally, for the creation of a plasma is also known the use of an electron cyclotron resonance (ECR) generator, which through the generation of microwaves coupled, on the one hand, to the generation of a magnetic field and, on the other hand, to the introduction of an appropriate gas will generate a cold plasma.

All these prior known solutions are not fully satisfactory as regards the result achieved, compared to the one expected and the means being implemented.

### SUMMARY OF THE INVENTION

Therefore, the objective of the present invention is to provide a new solution in order to cope, not only with the problem of preservation of the sterile nature of objects during their conditioning, but in addition to achieve a repetitive result in terms of sterilization quality thanks to optimized means being implemented.

Therefore, within the framework of an inventive step has been devised the combination of means for polarizing a support with a magnetic-field generator, in order to ensure both:
a sufficient molecular stirring of the gas contained in a container placed on the polarized support inside a sterilization vessel subjected to a secondary vacuum; and
an optimal concentration on this container of a flow of microwaves proceeding from an electron cyclotron resonance generator.

This finally permits to ignite the plasma only in the container and not in the vessel.

Thus, the invention relates to a device for cold-plasma sterilization for the sterilization of at least one object, such as a medical device, namely an implant, placed in a container containing at least one gas or a gas mixture appropriate for generating a cold plasma, said device comprising:
a closed vessel, in which is arranged a support for receiving said container;
means (5) for submitting the vessel to a secondary vacuum in the range from 10⁻³ to 10⁻⁷ millibar;

wherein the device also includes
an electron cyclotron resonance generator for generating in the vessel a flow of microwaves coupled to a magnetic field at least in a phase of initialization of the cold plasma;
means for polarizing said support of the container.

According to another feature of the invention, said device also includes at least one means for injecting into the container a gas or a gas mixture appropriate for generating a cold plasma at a pressure higher than the pressure in the sterilization vessel.

According to the invention, the container includes at least a first protection cap for injecting a gas or a gas mixture appropriate for generating a cold plasma.

According to another feature, said container also includes a second gas-exhaust protection cap.

According to yet another feature, the gas-injection means and/or the exhaust protection cap of the container constitute means for controlling a constant or nearly constant gas pressure in said container.

In particular, through this second protection cap the pressure in said container can be controlled so as to avoid its deterioration, while ensuring maintaining the cold plasma.

The invention also relates to a sterilization method implementing a sterilization device according to the invention, wherein:
at least one container containing at least one object, namely a medical device, is arranged on a support in a sterilization vessel;
said vessel is hermetically closed and it is subjected to a secondary vacuum;
the support of the container is subjected to a polarization, namely by radiofrequency;
through an ECR generator and at least in a phase of initialization of the cold plasma, the container is subjected to a flow of microwaves coupled to a magnetic field;
through the injection into the container of at least one gas or a gas mixture appropriate for generating a cold plasma, a pressure higher than the pressure in the sterilization vessel is maintained in the container.

According to another feature of the method according to the invention, after ignition of the cold plasma in the container, a constant pressure is maintained in the latter.

### BRIEF DESCRIPTION OF THE DRAWINGS

Further features and advantages of the invention will become clear from the following detailed description of the non-restrictive embodiments of the invention, with reference to the attached FIGURE.

FIG. 1 represents schematically a vertical cross-sectional view of an embodiment of a sterilization device according to the invention.

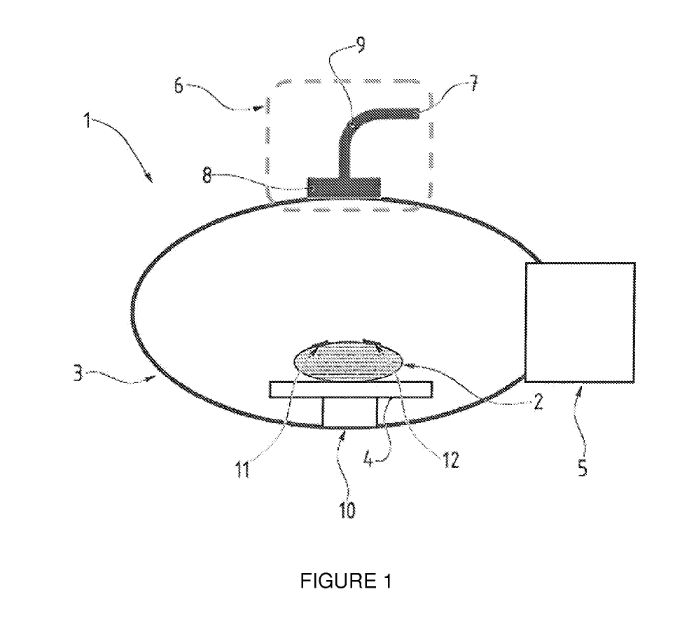

### DETAILED DESCRIPTION OF THE DRAWINGS

The present invention is related to the sterilization of objects, namely medical devices, such as surgical instruments, implants or the like. The invention relates in particular to the sterilization of these objects on the other hand contained in an adapted container corresponding, for example, to their packaging, and capable of enclosing at least one gas or a gas mixture appropriate for generating a cold plasma.

In this respect, it should be specified that though this container may, even prior to the sterilization operation, contain such a gas or gas mixture, the latter can also be injected during this sterilization.

The invention relates in particular to a cold-plasma sterilization device 1 for the sterilization of such objects arranged in a container 2.

Thus, this sterilization device 1 includes a closed sterilization vessel 3, however with appropriate accessing means, such as a door or an airlock, in order to be able to deposit in same the objects to be sterilized. Furthermore, in this vessel 3 is provided for an adapted support 4, for example in the form of a tray, for receiving the container 2 involved.

The vessel 3 is preferably tight so that during the plasma-generating process can be carried out the secondary vacuum inside. In particular, this vessel 3 should be capable of withstanding a pressure in the range from 10⁻³ to 10⁻⁷ millibar, i.e. in the range from 10⁻¹ to 10⁻⁵ Pascal (Pa), namely of 10⁻⁵ millibar, i.e. 10⁻³ Pascal (Pa), these values being within the range of the vacuum referred to as <<secondary vacuum>>.

In this respect, said device 1 also includes means 5 for subjecting the vessel 3 to such a secondary vacuum. For example, at one of the walls of this vessel 3 can be hermetically connected a vacuum-pumping device. Thus, before proceeding to a sterilization, the air contained in the vessel 3 is emptied following the introduction of the container 2 enclosing the object to be sterilized.

The device 1 also includes an ECR generator 6 or electron cyclotron resonance generator including microwave-generating means 7 and means for generating a magnetic field 8 in order to subject the container 2 in the vessel 3 to a flow of microwaves coupled to a magnetic field, at least in a phase of initialization of the cold plasma.

In particular, the microwave-generating means 7 include a waveguide 9 arranged so as to be capable of orienting and concentrating said microwaves emitted towards the vessel 3, and in particular towards the container 2.

According to an embodiment of the invention, this microwave generator 7 emits microwave having a power from a few tens to a few hundreds of Watts and preferably in the range of 250 W.

The means for generating a magnetic field 8 of the ECR 6 can, in turn, be formed of a solenoid provided with a coil of electric wires or permanent magnets and of multi-pole magnets operating in a traditional known way.

As can be seen in the FIGURE, these means for generating a magnetic field 8 surround the waveguide 9, which leads the microwaves into the vessel 3 above the support 4.

According to the invention, the sterilization device includes, in combination with the means explained above, means for polarizing 10 the support 4 at the container 2.

In particular, through this polarization results a molecular stirring in the container 2, which, combined with the focusing of the flow of microwaves generated by the ECR 6, has the advantage of ensuring the ignition of the cold plasma in the container 2 and not in the vessel, which is on the other hand subjected to a secondary vacuum.

Preferably, the polarization means 10 are of the type by radiofrequency, so as to excite the gas or gas mixture and thus to ensure the molecular stirring, which, through the focusing of the microwaves by magnetic field, leads to ionizing the gas and inducing the cold plasma in the container. By way of an example, this polarization by radiofrequency may have a power of some hundreds of Watts, preferably in the range of 250 Watt, with a polarization voltage in the range of 200 Volt.

It should be specified that the gas or gas mixture the container 2 must contain is of the plasmagene type. It can be formed of air or, in the case of a gas mixture, for example based on oxygen or argon.

In addition, the container 2 may contain such a plasmagene gas or gas mixture at the moment of its placing into the sterilization vessel 3 and/or it can be injected into same during the sterilization operation. As will be explained later, through such an injection during the sterilization process, it is possible to maintain in the container a sufficient gas pressure to maintain the cold plasma, which otherwise would extinguish too early and lead to an incomplete sterilization.

It should be noted that the container 2 can be formed of a bag made of polymeric material non-porous for the gas or the gas mixture it has to contain. Such a bag can also be designed according to specific standards depending on its contents. This is namely the case as regards the medical devices, implants or the like, fields to which the present invention is related in particular.

According to another feature of the invention, said sterilization device 1 can also include at least one means for injecting a gas or gas mixture appropriate for generating a cold plasma in the container 2 at a pressure higher than the pressure in the sterilization vessel 3. In fact, the container 2 preferably includes at least a first protective cap 11 in order to permit this gas injection. This gas-injection means can be formed of an injection nozzle connected to an adapted gas-distribution system.

In addition, said container 2 can be provided with at least one second gas-exhaust protective cap 12, in order to avoid its deterioration in case of overpressure, while ensuring the pressure necessary for maintaining the cold plasma in this container.

Advantageously, the gas-injection means and/or the exhaust protective cap 12 of the container 2 form means for controlling a constant or nearly constant gas pressure in said container.

The gas is preferably injected into said container 2 at a pressure lower than 100 Pa, taking into consideration that the sterilization vessel 3 is at a pressure lower than 10⁻³ Pa. This pressure difference between the sterilization vessel 3 and the inside of the container 2 will permit the induction of the plasma inside the container 2 while avoiding an eventual activation of a plasma in the vessel 3.

It should be noted that the gas or gas mixture can be injected continuously or discontinuously into the container 2 depending on the evolution of the gas flows in the container 2 during the reaction or also for purposes of maintenance of the plasma inside said container 2.

According to a preferred embodiment of the invention, the first gas-inlet protective cap 11, like the exhaust protective cap 12, is porous and designed so as to act as a valve, by permitting, for the first one 11, the injection of gas and, for the exhaust protective cap 12, as its name says, the exit of gas.

Advantageously, this second protective cap 12 also serves as a valve, which will permit to regulate the pressure inside said container during the sterilization procedure by expulsing a gas overflow, in order to impede the deterioration of this container, while avoiding the extinction of the plasma.

It is important to note that the flow rate of the gas or gas mixture injected in the container 2 depends on many parameters such as:
the pressure in the vessel 3;
the pressure in the container 2;
the volume of the container 2;
etc. . . .

According to an embodiment of the invention, the device 1 includes means for evaluating and regulating (not shown in the drawing) the pressure reigning inside the container 2. According to an exemplary embodiment, the means for evaluating the pressure can be formed of one or several optical sensors capable of following the evolution of the volume of the container, in this case when it is in the form of a bag, in order to determine and/or to estimate the pressure inside same. Obviously, it can also be contemplated to use other pressure sensors arranged on or in the container, eventually coupled to adapted wireless transmission means, in order to know this pressure in the container and to control the injection of gas. Likewise, one can also think of providing the gas-injection pipe, even a cannula connected to the exhaust protective cap 12 of the container 2, with such pressure-evaluation means.

As regards the regulation means, they can be in the form of a management unit capable of intervening, for example, on the means for injecting gas into the container depending on the information delivered to this management unit by said above-mentioned evaluation means.

The invention also relates to a sterilization method implementing such a sterilization device 1, consisting in that:
at least one container 2 enclosing at least one object, namely a medical device, is arranged on a support 4 in a sterilization vessel 3;
said vessel 3 is closed hermetically and is subjected to a secondary vacuum;
the support 4 in the container 2 is subjected to a polarization, namely by radiofrequency;
through an ECR generator 6 and at least in a phase of initialization of the cold plasma, the container 2 is subjected to a flow of microwaves coupled to a magnetic field;
by injecting into the container 2 at least one gas or an appropriate gas mixture for generating a cold plasma, a pressure higher than the pressure in the sterilization vessel 3 is maintained in the container 2.

Advantageously, after ignition of the cold plasma in the container 2, in the latter is maintained a constant pressure, i.e. a pressure stable, or substantially constant, at a given value, i.e. varying by more or less 1 to 10% around said given value.

Thus, the combined effect of a microwave generator 7 coupled to a magnetic field induced by the generating means 8 as well as the polarization by radiofrequency of the support 4 on which said container 2 rests leads to <<igniting>< the plasma only in the container 2 and not in the sterilization vessel 3, which is subjected to a secondary vacuum.

1. Device for cold-plasma sterilization of at least one object, said device comprising:
a container holding the object and a gas mixture appropriate for generating a cold plasma;
a closed vessel having a support for receiving said container arranged in said closed vessel;
means for submitting the vessel to a secondary vacuum in a range from 10⁻³ to 10⁻⁷ millibar;
an electron cyclotron resonance generator for generating in the vessel a flow of microwaves coupled to a magnetic field at least in a phase of initialization of the cold plasma; and
means for polarizing said support of the container.

2. Device according to claim 1, wherein the polarization means are of a type by radiofrequency.

3. Device according to claim 1, further comprising:
at least one means for injecting a gas appropriate for generating a cold plasma into said container at a pressure higher than a pressure in the vessel.

4. Device according to claim 1, wherein the container comprises at least a first protective cap for injecting a gas appropriate for generating a cold plasma.

5. Device according to claim 4, wherein said container further comprises at least one second gas-exhaust protective cap.

6. Device according to claim 3, further comprising:
means for controlling a gas pressure in said container, being comprised of the gas-injection means, a exhaust protective cap of the container, or both.

7. Device according to claim 1, further comprising:
means for evaluating and regulating the pressure reigning inside the container.

8. Sterilization method implementing a sterilization device according to claim 1, the method comprising the steps of:
enclosing at least one object in at least one container, said at least one object being arranged on a support in a sterilization vessel;
hermetically closing the vessel and subjecting the vessel to a secondary vacuum;
polarizing the support in the container by radiofrequency;
subjecting the container to a flow of microwaves coupled to a magnetic field through an electron cyclotron resonance generator and at least in a phase of initialization of the cold plasma; and
injecting at least one gas into the container for generating a cold plasma, a pressure higher than a pressure in the vessel being maintained in said container.

9. Sterilization method according to claim 8, wherein, after ignition of the cold plasma in the container, a constant pressure is maintained in the container.
